(19) United States
(12) Patent Application Publication
(10) Pub. No.: US 2003/0104050 A1
(43) Pub. Date: Jun. 5, 2003

(54) Process for preparing quick dissolving, high loading ribavirin compositions

(76) Inventors: Amol Singh Matharu, Bedminster, NJ (US); Mahendra R. Patel, East Brunswick, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/285,188

(22) Filed: Oct. 31, 2002

Related U.S. Application Data
(60) Provisional application No. 60/336,853, filed on Nov. 2, 2001.

(51) Int. Cl. A61K 31/7056; A61K 9/48; A61K 9/20

(52) U.S. Cl. 424/465; 514/43

(57) ABSTRACT

A quick dissolving, high loading ribavirin composition which is essentially free of ribavirin polymorphic forms, said composition is prepared by a process comprising: (a) mixing ribavirin, at least one disintegrant and/or at least one filler to form an homogenous mixture; (b) granulating the mixture in the presence of water to form a granulation; (c) drying the granulation; and (d) mixing at least one disintegrant and/or at least one lubricant with the granulation to produce a ribavirin composition, wherein said ribavirin composition comprises at least 80 weight percent of ribavirin, based on the total weight of the composition. The ribavirin compositions prepared by the wet granulation process of the invention are free flowing and have adequate bulk density for processing into capsules. In addition, the ribavirin compositions contain at least 80 weight percent of ribavirin. Furthermore, the ribavirin compositions are substantially free of polymorphic forms of ribavirin, i.e., there are no signs of polymorphic change in ribavirin as determined by infrared spectrophotometry.

### FIELD OF THE INVENTION

The present invention relates to a quick dissolving ribavirin composition which comprises at least 80% by weight ribavirin and is prepared by a wet granulation process.

### BACKGROUND OF THE INVENTION

Ribavirin (C₈H₁₂N₄O₅) is an antiviral agent which has been administered alone or in combination with interferon alpha-2b to treat patients with chronic hepatitis C infections. Ribavirin is commercially available in capsule form under the trademarks Virazole® and Rebetol®. U.S. Pat. Nos. 6,051,252; 5,916,594; and 5,914,128 describe ribavirin compositions prepared by a dry compacting method at a compressing force from about 50 to about 75 kN. Such a high compressing force is necessary due to the poor compressibility of ribavirin. However, heat is produced during the dry compacting operation which may cause the formation of ribavirin polymorphic forms. Such polymorphic forms are undesirable.

U.S. Pat. No. 4,439,453 describes a wet granulation method for granulating acetaminophen which involves blending acetaminophen powder or crystals with excipients; wetting to a moist powder consistency with an aqueous binder solution; drying; milling; and then blending with more excipients and a lubricant. The wet granulation process requires a large amount of excipients, generally from about 25 to about 40 weight percent, based on the dried weight of the pharmaceutical composition. The advantages of wet granulation as compared to roller compaction process are that wet granulation: (1) provides the material to be compressed with better wetting properties, particularly in the case of hydrophobic drug substances; (2) allows the use of a hydrophilic excipient in the process which makes the surface of a hydrophobic drug more hydrophilic which improves disintegration and dissolution; (3) improves content uniformity of the solid dosage forms because the granules obtained thereby usually contain approximately the same amount of ingredients, and thus, segregation of different ingredients of the material to be compressed due to different physical characteristics such as density is avoided; and (4) optimizes the particle size and shape of particles by creating approximately spherical granules.

Thus, there is a need for a granulated ribavirin composition which is quick dissolving, free flowing, and has adequate bulk density for processing into capsules. A further need is for such a granulation composition to provide a high load, for example, at least 80%, of ribavirin in the composition. In addition, the ribavirin composition should be substantially free of polymorphic forms of ribavirin.

### SUMMARY OF THE INVENTION

The invention provides a quick dissolving, high loading ribavirin composition which is essentially free of ribavirin polymorphic forms, said composition is prepared by a process comprising: (a) mixing ribavirin, at least one disintegrant and/or at least one filler to form an homogenous mixture; (b) granulating the mixture in the presence of water to form a granulation; (c) drying the granulation; and (d) mixing at least one disintegrant and/or at least one lubricant with the granulation to produce a ribavirin composition, wherein said ribavirin composition comprises at least 80 weight percent of ribavirin, based on the total weight of the composition.

According to another aspect, the invention provides a quick dissolving, high loading ribavirin composition which is essentially free of ribavirin polymorphic forms, said composition is prepared by a process comprising: (a) mixing ribavirin, at least one disintegrant and/or at least one filler to form an homogenous mixture; (b) granulating the mixture in the presence of a solvent to form a granulation; (c) drying the granulation; (c′) milling the dried granulation; and (d) mixing at least one disintegrant and/or at least one lubricant with the milled granulation to produce a ribavirin composition, wherein said ribavirin composition comprises at least 80 weight percent of ribavirin, based on the total weight of the composition.

The ribavirin compositions prepared by the wet granulation process of the invention are uniform, free flowing, and have adequate bulk density for processing into capsules. In addition, the ribavirin compositions contain at least 80 weight percent of ribavirin. Furthermore, the ribavirin compositions are substantially free of polymorphic forms of ribavirin, i.e., there are no signs of polymorphic change in ribavirin as determined by infrared spectrophotometry.

### DESCRIPTION OF THE INVENTION

The ribavirin compositions of the invention are quick dissolving. As used herein, “quick dissolving” means that about 90% of the ribavirin from the compositions of the invention is consistently dissolved in water in 15 minutes and about 100% of the ribavirin from the compositions of the invention is dissolved in water in about 30 minutes.

The ribavirin compositions preferably contain at least 80 weight percent of ribavirin, based on the total weight of the composition. More preferably, the compositions contain at least 90 weight percent of ribavirin. The ribavirin compositions are prepared by a wet granulation process. In the first step, step (a), ribavirin, at least one disintegrant and/or at least one filler are mixed to form an homogenous mixture.

Examples of disintegrants include pharmaceutically acceptable disintegrants which are chemically and physically compatible with ribavirin. Preferred disintegrants are selected from croscarmellose sodium, hydroxypropyl cellulose, hydroxypropylmethyl cellulose, sodium starch glycolate, corn starch, pregelatinized starches, polacrillin potassium, polyacrylates such as Carbopol®, sodium carboxymethyl cellulose, potato starch, microcrystalline cellulose, cross-linked polyvinylpyrrolidone, magnesium aluminium silicate, bentonite, alginic acid and alginates. A combination of disintegrants may also be used. A preferred disintegrant is croscarmellose sodium.

The amount of disintegrant in the compositions of the invention is preferably from about 0.1 to about 10 weight percent, based on the total weight of the ribavirin composition. More preferably, the amount of disintegrant in the ribavirin compositions is from about 2 to about 8 weight percent, most preferably about 3 to about 6 weight percent.

Examples of fillers include any pharmaceutically acceptable filler which provides bulk to the ribavirin composition and which is physically and chemically compatible with ribavirin. Preferred fillers are selected from lactose anhydrous, microcrystalline cellulose, starch, pregelatinized starch, modified starch, dibasic calcium phosphate dihydrate, calcium sulfate trihydrate, calcium sulfate dihydrate, calcium carbonate, lactose, dextrose, sucrose, mannitol, and sorbitol. A combination of fillers may also be used. Preferred fillers are mannitol and lactose monohydrate.

The amount of filler in the compositions of the invention is preferably from about 1 to about 20 weight percent, based on the total weight of the ribavirin composition. More preferably, the amount of filler in the ribavirin compositions is from about 5 to about 17 weight percent, most preferably about 10 to about 15 weight percent.

In the second step, step (b), a solvent is added to the homogenous mixture prepared in the first step to form a granulation. Suitable solvents include, but are not limited to, water, acetonitrile, ethyl acetate, acetone, benzene, toluene, dioxane, dimethylformamide, chloroform, methylene chloride, ethylene chloride, carbon tetrachloride, chlorobenzene, acetone, methanol, ethanol, isopropanol, and butanol. A combination of solvents may also be used. Preferably, the solvent is water.

In the third step, step (c), the granulation prepared in the second step is dried. Drying is preferably performed using a VMA 1200 Vega drier. Optionally, a milling step is employed in the process following drying. A preferred milling device is a BTS 200 high speed mill equipped with 1.1 mm.

In the forth step, step (d), at least one disintegrant and/or at least one lubricant is mixed with either the dried granulation prepared in step (c) or with the milled granulation prepared in step (c′). A preferred means of mixing the lubricant with the dried granulation is with a BIN blender. Optionally, a pre-lubrication step may be employed wherein the dried granulation is mixed with a disintegrant prior to mixing with a lubricant.

Examples of lubricants include any pharmaceutically acceptable solid or liquid lubricant which is used to enhance the flow and prevent sticking of the ribavirin composition after granulation and which is chemically and physically compatible with ribavirin. Preferred lubricants are selected from magnesium stearate, calcium stearate, zinc stearate, talc, propylene glycol, polyethylene glycol, stearic acid, vegetable oil, sodium benzoate, sodium lauryl sulfate, magnesium lauryl sulfate, mineral oil, and polyoxyethylene monostearate. A combination of lubricants may also be used. A preferred lubricant is magnesium stearate.

The amount of lubricant in the compositions of the invention is preferably from about 0.1 to about 5 weight percent, based on the total weight of the ribavirin composition. More preferably, the amount of lubricant in the ribavirin compositions is from about 0.5 to about 2 weight percent, most preferably about 0.8 to about 1.8 weight percent.

It is within the scope of the invention that a specific compound used in the process of the invention may have more than one function. For example, a disintegrant may also function as a lubricant, or vice versa, etc. In the case where a compound functions in at least a dual manner with respect to the critical components of the process, disintegrant, filler, and lubricant, other than ribavirin, the amount of such compound should be at least the minimum additive amount required for the respective functions.

The ribavirin compositions of the invention are preferably in the form of a capsule. In a preferred embodiment, the ribavirin compositions are encapsulated. A preferred encapsulating device is a MG2 Suprema capsule filling machine equipped with a size 2 change part.

The amount of ribavirin in the compositions of the invention is preferably from about 50 mg to 2500 mg. More preferably, the amount of ribavirin in the compositions is from 200 mg to 400 mg.

In addition to the ribavirin, disintegrant, filler, and lubricant, the compositions of the invention may contain additional ingredients. Such additional ingredients include natural and/or artificial ingredients which are commonly used to prepare pharmaceutical compositions. Examples of additional ingredients include enteric coating agents, diluents, binders, anti caking agents, amino acids, fibers, solubilizers, emulsifiers, flavorants, enzymes, buffers, stabilizers, colorants, dyes, antioxidants, anti-adherents, preservatives, electrolytes, glidants, and carrier materials. A combination of additional ingredients may also be used. Such ingredients are known to those skilled in the art, and thus, only a limited number will be specifically referenced.

Examples of enteric coating agents include: hydroxypropylmethylcellulose phthalate, methacrylic acid-methacrylic acid ester copolymer, polyvinyl acetate-phthalate and cellulose acetate phthalate.

Examples of binders include: starches, e.g., potato starch, wheat starch, corn starch; gums such as gum tragacanth, acacia gum, and gelatin; microcrystalline cellulose, e.g., products known under the registered trademarks Avicel, Filtrak, Heweten or Pharmacel, hydroxypropyl cellulose, hydroxyethyl cellulose, and hydroxypropylmethyl cellulose; and polyvinyl pyrrolidone, e.g., Povidone.

Examples of glidants include: silica, magnesium trisilicate, powdered cellulose, starch, talc and tribasic calcium phosphate. Colloidal silica (e.g., Aerosil) is particularly preferred.

Examples of solubilizers and/or emulsifiers include: sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20.

The invention is further defined by reference to the following examples, which are intended to be illustrative and not limiting.

### EXAMPLE 1

### Preparation of a Ribavirin Composition

A pre-mix was prepared which contained 200 mg of ribavirin, 45 mg of mannitol, 10 mg of Phamacoat 606, and 10 mg of Povidone K-90. Deionized water was added to the pre-mix to form a granulation. The granulation was dried using a VMA 1200 Vega drier. The dried granulation was milled using a BTS 200 High Speed mill equipped with 1.1 mm screen. Crosscarmalose sodium, 12.5 mg, which was screened through a hand screen #20, was added to the milled granulation and mixed using a PM 1000 Bin Blender. Magnesium Stearate, 2.5 mg, was added and mixed using a PM 1000 Bin Blender to form a final mix. Total weight of powder=280 mg.

### EXAMPLE 2

### Evaluation of Ribavirin Composition

Three different trials were conducted (Trials I, II, and III) using the ribavirin composition prepared.

In Example 1. In each trial a random sample of the ribavirin composition prepared in Example 1 was analyzed for bulk density and tap density. As used herein, “tap density” means the measured mass of a powder attained at a limiting volume measured in a cylinder after being “tapped down”, typically by a mechanical device. The tap density is measured in accordance with the procedure described in USP 23, NF 18, Supplement 6 (1997), procedure No. 616 at page 3768. The test results are summarized in Table I.

TABLE I

| | | Bulk | Tap |
|---|---|---|---|
| Ribavirin | Ribavirin Composition | Density | Density |
| Composition | Blend Location | (g/mL) | (g/mL) |
| Trial I | Top Center | 0.5 | 0.63 |
| | Middle Center | 0.51 | 0.63 |
| | Bottom Center | 0.51 | 0.63 |
| | Composition Sample | 0.5 | 0.63 |
| Trial II | Top Center | 0.52 | 0.7 |
| | Middle Center | 0.53 | 0.7 |
| | Bottom Center | 0.53 | 0.67 |
| | Composition Sample | 0.54 | 0.68 |
| Trial III | Top Center | 0.53 | 0.67 |
| | Middle Center | 0.54 | 0.69 |
| | Bottom Center | 0.54 | 0.67 |
| | Composition Sample | 0.53 | 0.67 |

The results in Table I clearly show that the ribavirin composition prepared in Example 1 according to the wet granulation process of the invention was uniform in terms of tap density and has adequate bulk density for processing into capsules. In addition, the ribavirin composition contained 80 weight percent of ribavirin.

The ribavirin composition prepared in Example 1 was analyzed using a infrared spectrophotometer which showed no signs of polymorphic change in the ribavirin.

While the invention has been described with particular reference to certain embodiments thereof, it will be understood that changes and modifications may be made by those of ordinary skill within the scope and spirit of the following claims:

What is claimed is:

1. A quick dissolving, high loading ribavirin composition which is essentially free of ribavirin polymorphic forms, said composition is prepared by a process comprising: (a) mixing ribavirin, at least one disintegrant and/or at least one filler to form an homogenous mixture; (b) granulating the mixture in the presence of water to form a granulation; (c) drying the granulation; and (d) mixing at least one disintegrant and/or at least one lubricant with the granulation to produce a ribavirin composition, wherein said ribavirin composition comprises at least 80 weight percent of ribavirin, based on the total weight of the composition.

2. A quick dissolving, high loading ribavirin composition which is essentially free of ribavirin polymorphic forms, said composition is prepared by a process comprising: (a) mixing ribavirin, at least one disintegrant and/or at least one filler to form an homogenous mixture; (b) granulating the mixture in the presence of water to form a granulation; (c) drying the granulation; (c′) milling the dried granulation; and (d) mixing at least one disintegrant and/or at least one lubricant with the milled granulation to produce a ribavirin composition, wherein said ribavirin composition comprises at least 80 weight percent of ribavirin, based on the total weight of the composition.

3. The composition according to claim 1 wherein the solvent is selected from the group consisting of water, acetonitrile, ethyl acetate, acetone, benzene, toluene, dioxane, dimethylformamide, chloroform, methylene chloride, ethylene chloride, carbon tetrachloride, chlorobenzene, acetone, methanol, ethanol, isopropanol, butanol, and combinations thereof.

4. The composition according to claim 3 wherein the solvent is water.

5. The composition according to claim 1 wherein the disintegrant is selected from the group consisting of croscarmellose sodium, hydroxypropyl cellulose, hydroxypropylmethyl cellulose, sodium starch glycolate, corn starch, pregelatinized starches, polacrillin potassium, polyacrylates, sodium carboxymethyl cellulose, potato starch, microcrystalline cellulose, cross-linked polyvinylpyrrolidone, magnesium aluminium silicate, bentonite, alginic acid, alginates, and combinations thereof.

6. The composition according to claim 4 wherein the disintegrant is croscarmellose sodium.

7. The composition according to claim 1 wherein the disintegrant is present in an amount of from about 0.1 to about 10 weight percent, based on the total weight of the ribavirin composition.

8. The composition according to claim 7 wherein the disintegrant is present in an amount of from about 2 to about 8 weight percent.

9. The composition according to claim 8 wherein the disintegrant is present in an amount of from about 3 to about 6 weight percent.

10. The composition according to claim 1 wherein the filler is selected from the group consisting of lactose anhydrous, microcrystalline cellulose, starch, pregelatinized starch, modified starch, dibasic calcium phosphate dihydrate, calcium sulfate trihydrate, calcium sulfate dihydrate, calcium carbonate, lactose, dextrose, sucrose, mannitol, sorbitol, and combinations thereof.

11. The composition according to claim 10 wherein the filler is mannitol or lactose monohydrate.

12. The composition according to claim 1 wherein the filler is present in an amount of from about 1 to about 20 weight percent, based on the total weight of the ribavirin composition.

13. The composition according to claim 12 wherein the filler is present in an amount of from about 5 to about 17 weight percent.

14. The composition according to claim 13 wherein the filler is present in an amount of from about 10 to about 15 weight percent.

15. The composition according to claim 1 wherein the lubricant is selected from the group consisting of magnesium stearate, calcium stearate, zinc stearate, talc, propylene glycol, polyethylene glycol, stearic acid, vegetable oil, sodium benzoate, sodium lauryl sulfate, magnesium lauryl sulfate, mineral oil, polyoxyethylene monostearate, and combinations thereof.

16. The composition according to claim 15 wherein the lubricant is magnesium stearate.

17. The composition according to claim 1 wherein the lubricant is present in an amount of from about 0.1 to about 5 weight percent, based on the total weight of the ribavirin composition.

18. The composition according to claim 17 wherein the lubricant is present in an amount of from about 0.5 to about 2 weight percent.

19. The composition according to claim 18 wherein the lubricant is present in an amount of from about 0.8 to about 1.8 weight percent.

20. The composition according to claim 1 wherein the process additionally comprises an encapsulation step.

21. The composition according to claim 1 which is in the form of a capsule.

22. The composition according to claim 1 wherein the ribavirin is present in an amount of at least 90 weight percent.
